(19) United States
(12) Patent Application Publication
(10) Pub. No.: US 2025/0216220 A1
(43) Pub. Date: Jul. 3, 2025

(54) METHODS AND SYSTEMS FOR DETERMINING A STEP COUNT OF A USER

(71) Applicant: SAMSUNG ELECTRONICS CO., LTD., Suwon-si (KR)

(72) Inventors: Amod Ashok CHOURASIA, Nagpur (IN); Rahul Dewangan, Raigarh (IN)

(73) Assignee: SAMSUNG ELECTRONICS CO., LTD., Suwon-si (KR)

(21) Appl. No.: 19/084,344

(22) Filed: Mar. 19, 2025

Related U.S. Application Data
(63) Continuation of application No. PCT/KR2023/013945, filed on Sep. 15, 2023.

(30) Foreign Application Priority Data
Sep. 20, 2022 (IN) 202211053818

(51) Int. Cl. G01C 22/00; A61B 5/11

(52) U.S. Cl. CPC G01C 22/006; A61B 5/112

(57) ABSTRACT

A method includes: determining a motion pattern of a user after an initiation of a walking activity; detecting one or more false steps and a gait abnormality associated with the user, based on the motion pattern; detecting an arm swing angle of the user and a leg swing angle of the user, based on the motion pattern and the walking activity; estimating a first variation in the arm swing angle and a second variation in the leg swing angle in the walking activity; calculating, using a machine learning (ML) model of a plurality of ML models, a compensation value associated with the one or more false steps, based on a combination of the first variation and the second variation, and a height of the user; and determining a step count of the user, based on the calculated compensation value and an initial step count of the user.

### CROSS-REFERENCE TO RELATED APPLICATIONS

This application is a by-pass continuation application of International Application No. PCT/KR2023/013945, filed on Sep. 15, 2023, which is based on and claims priority to Indian Patent Application number 202211053818, filed on Sep. 20, 2022, in the Indian Intellectual Property Office, the disclosures of which are incorporated by reference herein their entireties.

### BACKGROUND

### 1. Field

The disclosure generally relates to step counting and particularly relates to a method and a system for determining a step count of a user with a physical disability.

### 2. Description of Related Art

Traditionally, a user requires mobile devices applications that allow more comprehensive personal health monitoring using device sensors and smart wearables that monitor physical activities to provide the user a comprehensive and easy way to keep track on their health and creating a healthy lifestyle.

Among all physical activities, walking is the most widely performed. The number of steps taken is widely recognized as a simple and objective indicator of the amount of walking that has been performed.

For counting steps, a number of methods may be implemented. The most common and accurate methods are based on either peak detection or threshold crossing. In peak detection of accelerometer signals for step counting, a step is detected whenever a peak is detected in combined magnitude. In threshold crossing detection of accelerometer signals, a step is detected whenever the accelerometer signal crosses threshold. Threshold is the average of the maximum and minimum values from the previous.

A factor associated with walking is a gait (variable step sizes of the user's walking) that may be defined as a manner of walking. Normal walking gait includes more continuous regular peaks pattern. Crutch walking includes multiple peaks clustered with some time gap indicating slow walking with instability. In crutch walking, steps may not be detected due to time gap and irregularity.

Furthermore, distance walked, and calories burned for a user walking normally and another user walking with a smaller step size are detected as the same irrespective of step length as the distance is estimated on basis of height manually inputted by user.

In a conventional system, while user moves with the support of accessories such as crutches or walkers, their movement is not as a normal human being, which is due to deficiencies in walking or running style. These deficiencies occur due to following reasons:
a. Abnormality/Disorder in Gait [Issue in legs/feet/Knee/Ankle, injury in leg]
b. Variable step length [with crutches/walkers]

Therefore, there is a need for a method to count or approximate steps for such persons.

### SUMMARY

Provided are a method and a system for determining a step count of a user.

Provided are a method and a system for determining a step count of a user.

According to an aspect of the disclosure, a method for determining a step count of a user, includes: determining a motion pattern of the user after an initiation of a walking activity by the user; detecting one or more false steps and a gait abnormality associated with the user, based on the motion pattern; detecting an arm swing angle of the user and a leg swing angle of the user, based on the motion pattern and the walking activity; estimating a first variation in the arm swing angle and a second variation in the leg swing angle in the walking activity; calculating, using a machine learning (ML) model of a plurality of ML models, a compensation value associated with the one or more false steps, based on a combination of the first variation and the second variation, and a height of the user; and determining the step count of the user, based on the calculated compensation value and an initial step count of the user.

According to an aspect of the disclosure, a system for determining a step count of a user, includes: a determination engine configured to determine a motion pattern of a user after initiation of a walking activity by the user; an abnormality detection engine configured to detect one or more false steps and a gait abnormality associated with the user, based on the motion pattern; a swing classification engine configured to: detect an arm swing angle of the user and a leg swing angle of the user, based on the motion pattern in the walking activity; and estimate a first variation in the arm swing angle and a second variation in the leg swing angle in the walking activity; a compensation value calculation engine configured to calculate, using a Machine Learning (ML) model of a plurality of ML models, a compensation value associated with the one or more false steps, based on a combination of the first variation and the second variation, and a height of the user; and a step detection engine configured to determine the step count of the user, based on the calculated compensation value and an initial step count of the user.

These aspects and advantages will be more clearly understood from the following detailed description taken in conjunction with the accompanying drawings and claims.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 illustrates a block diagram depicting a method for determining a step count of a user, in accordance with an embodiment of the disclosure;

FIG. 2 illustrates a system configured to determine a step count of the user, in accordance with an embodiment of the disclosure;

FIG. 3 illustrates a process for determining a step count of a user, in accordance with an embodiment of the disclosure;

FIG. 4 illustrates a method for determining a step count of a user, in accordance with an embodiment of the disclosure;

FIG. 5 illustrates a process for determining a gait abnormality associated with the user, in accordance with an embodiment of the disclosure;

FIG. 6A illustrates a process for determining an arm swing angle of a user and a leg swing angle of a user, in accordance with an embodiment of the disclosure;

FIG. 6B illustrates a block diagram of a crutch cycle of a user during the walking activity, in accordance with an embodiment of the disclosure;

FIG. 7 illustrates a process for determining a first pre-determined threshold value associated with an arm swing angle and a second pre-determined threshold value associated with a leg swing angle, in accordance with an embodiment of the disclosure;

FIG. 8 illustrates a process for detecting a physical aid used by a user during a walking activity, in accordance with an embodiment of the disclosure;

FIG. 9 illustrates a process for calculating a compensation value associated with one or more false steps of a user, in accordance with an embodiment of the disclosure; and

FIG. 10 illustrates a process for determining the step count of a user based on a compensation value, in accordance with an embodiment of the disclosure.

### DETAILED DESCRIPTION

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are examples of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skilled in the art to which this disclosure belongs. The system, methods, and examples provided herein are illustrative only and not intended to be limiting.

Embodiments of the disclosure are described below in detail with reference to the accompanying drawings.

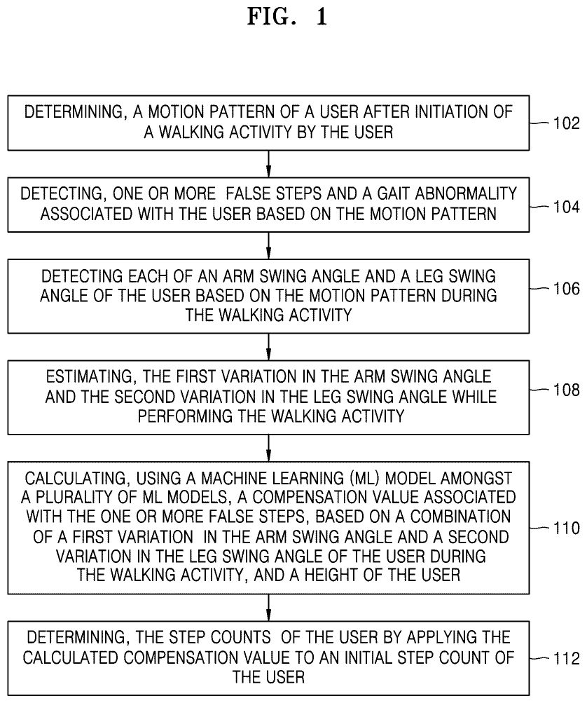

FIG. 1 illustrates a block diagram depicting a method for determining a step count of a user, in accordance with an embodiment of the disclosure. The method may be implemented in an electronic device. Examples of the electronic device may include, but are not limited to, a smartphone, a smart watch, a laptop, a tablet, or the like. The method may include determining the step count of the user by employing a Machine Learning (ML) technique. Further, the user may perform the walking activity by using a physical aid.

At block 102, the method includes determining a motion pattern of a user after initiation of a walking activity by the user. The determining of the motion pattern may be performed by a determination engine included in an electronic device or a system.

At block 104, the method includes detecting one or more false steps and a gait abnormality associated with the user based on the motion pattern. The detecting of the one or more false steps and the gait abnormality may be performed by an abnormality detection engine included in an electronic device or a system.

At block 106, the method includes detecting each of an arm swing angle and a leg swing angle of the user based on the motion pattern during the walking activity. The detecting of the arm swing angle and the leg swing angle may be performed by a swing classification engine included in an electronic device or a system.

At block 108, the method includes estimating a first variation in the arm swing angle and a second variation in the leg swing angle while performing the walking activity. The estimating of the first variation and the second variation may be performed by the swing classification engine included in an electronic device or a system.

At block 110, the method includes calculating using a Machine Learning (ML) model amongst a plurality of ML models, a compensation value associated with the one or more false steps, based on a combination of a first variation in the arm swing angle and a second variation in the leg swing angle of the user during the walking activity, and a height of the user. The calculating of the compensation value may be performed by a compensation value calculation engine included in an electronic device or a system.

At block 112, the method includes determining the step counts of the user during walking activity by applying the calculated compensation value to an initial step count of the user. The determining of the step counts of the user may be performed by a step detection engine included in an electronic device or a system.

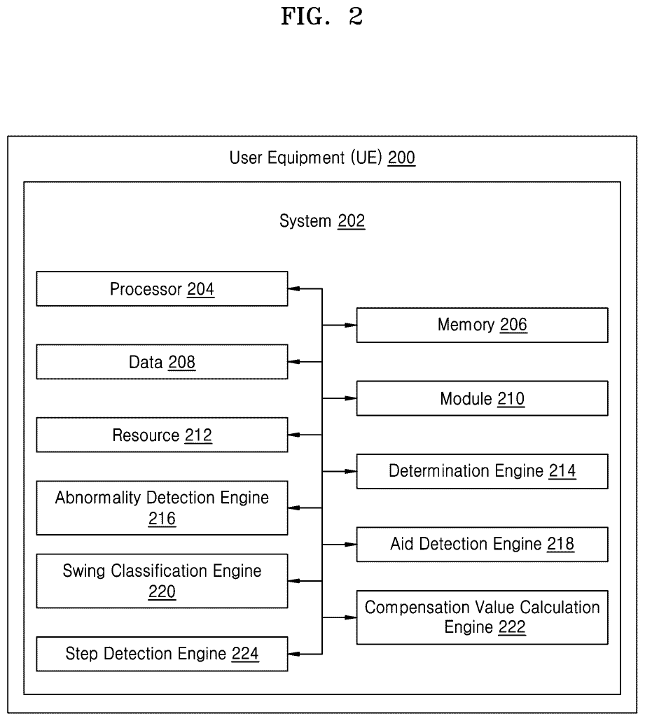

FIG. 2 illustrates a schematic block diagram 200 of a system 202 configured to determine a step count of the user, in accordance with an embodiment of the disclosure. In an embodiment, the system 202 may be incorporated in an electronic device. Examples of the electronic device may include, but are not limited to, a smartphone, a smart watch, a laptop, and a tablet. In an embodiment, the system 202 may be configured to employ a ML model for determining the step count of the user. The system 202 may be configured to determine the step count of the user after the initiation of a walking activity by the user. Further, the user may be walking with an assistance of a physical aid.

In one example embodiment, the system 202 can be a chip incorporated in the electronic device. In one example embodiment, the system 202 may be an implemented software, a logic-based program, a hardware, a configurable hardware, or the like. The system 202 may include a processor 204, a memory 206, data 208, module(s) 210, resource(s) 212, a determination engine 214, an abnormality detection engine 216, a swing classification engine 220, an aid detection engine 218, a compensation value calculation engine 222, and a step detection engine 224.

The processor 204, the memory 206, the data 208, the module(s) 210, the resource(s) 212, the determination engine 214, the abnormality detection engine 216, the swing classification engine 220, the aid detection engine 218, the compensation value calculation engine 222, and the step detection engine 224 may be communicatively coupled to one another.

In an example, the processor 204 may be a single processing unit or a number of units, all of which could include multiple computing units. The processor 204 may be implemented as one or more microprocessors, microcomputers, microcontrollers, digital signal processors, central processing units, processor cores, multi-core processors, multiprocessors, state machines, logic circuitries, application-specific integrated circuits, field-programmable gate arrays and/or any devices that manipulate signals based on operational instructions. Among other capabilities, the processor 204 may be configured to fetch and/or execute computer-readable instructions and/or data stored in the memory 206.

In an example, the memory 206 may include any non-transitory computer-readable medium known in the art including, for example, volatile memory, such as static random-access memory (SRAM) and/or dynamic random-access memory (DRAM), and/or non-volatile memory, such as read-only memory (ROM), erasable programmable ROM (EPROM), flash memory, hard disks, optical disks, and/or magnetic tapes. The memory 206 may include the data 208.

The data 208 serves, amongst other things, as a repository for storing data processed, received, and generated by one or more of the processor 204, the module(s) 210, the resource(s) 212, the determination engine 214, the abnormality detection engine 216, the swing classification engine 220, the aid detection engine 218, the compensation value calculation engine 222, and the step detection engine 224.

The module(s) 210, amongst other things, may include routines, programs, objects, components, data structures, etc., which perform particular tasks or implement data types. The module(s) 210 may also be implemented as, signal processor(s), state machine(s), logic circuitries, and/or any other device or component that manipulate signals based on operational instructions.

Further, the module(s) 210 may be implemented in hardware, instructions executed by at least one processing unit, for example, the processor 204, or by a combination thereof. The processing unit may be a general-purpose processor which executes instructions to cause the general-purpose processor to perform operations or, the processing unit may be dedicated to performing the required functions. In one aspect of the disclosure, the module(s) 210 may be machine-readable instructions (software) which, when executed by a processor 204 or processing unit, may perform any of the described functionalities.

The resource(s) 212 may be physical and/or virtual components of the system 202 that provide inherent capabilities and/or contribute towards the performance of the system 202. Examples of the resource(s) 212 may include, but are not limited to, a memory (e.g., the memory 206), a power unit (example, a battery), a display unit, etc. The resource(s) 212 may include a power unit/battery unit, a network unit, etc., in addition to the processor 204, and the memory 206.

Continuing with the above embodiment, the determination engine 214 may be configured to determine a motion pattern of a user. The motion pattern of the user may be determined after initiation of the walking activity by the user. The motion of the user may indicate a posture of the user while walking, hand moments, feet moments, and a size for each step taken by the user after initiating the walking activity. The initiation of the walking activity may be detected by one or more sensors. Further, the one or more sensors may notify the determination engine 214 about the initiation. Examples of the one or more sensors may include, an accelerometer, and a gyroscope. Furthermore, the one or more sensors may be incorporated within the electronic device. In an embodiment, the one or more sensors may be within a vicinity of the user.

Subsequent to determination of the motion pattern by the determination engine 214, the abnormality detection engine 216 may be configured to detect one or more false steps taken by the user after initiating the walking activity and the gait abnormality associated with the user. The one or more false steps and the gait abnormality may be detected based on the motion pattern of the user while performing the walking activity. Examples of the gait abnormality may include, but are not limited to, a spastic gait, a scissors gait, a steppage gait, a waddling gait, a propulsive gait, an abnormality due to an amputation, and a biological abnormality.

In one embodiment, the aid detection engine 218 may be configured to detect the physical aid used by the user during the walking activity based on the detection of the gait abnormality. Examples of the physical aid may include, but are not limited to, a crutch, a leg brace, a walker, and canes.

Subsequently, the swing classification engine 220 may be configured to detect an arm swing angle of the user and a leg swing angle of the user while performing the walking activity. The arm swing angle may be detected for one or more arms of the user and the legs swing angle may be detected for one or more legs of the user. The arm swing angle and the leg swing angle may be detected based on the motion pattern of the user, the gait abnormality associated with the user, the walking aid used by the user, and sensor data received from the one or more sensors.

Continuing with the above embodiment, upon detecting the arm swing angle and the leg swing angle, the swing classification engine 220 may further be configured to determine a first pre-determined threshold value associated with the arm swing angle and a second pre-determined threshold value associated with the leg swing angle. The first pre-determined threshold value and the second pre-determined threshold value may be determined based on a height of the user, and the physical aid assisting the user while performing the walking activity. The height of the user may be received as input from the user at an interface of the electronic device.

Subsequently, the swing classification engine 220 may be configured to determine whether the arm swing angle is less than the first pre-determined threshold value and the leg swing angle is less than the second pre-determined threshold value. In an embodiment, where it is determined that the arm swing angle is less than the first pre-determined threshold value and the leg swing angle is less than the second pre-determined threshold value, a first variation in the arm swing angle and a second variation in the leg swing angle may be estimated to further calculate a compensation value associated with the one or more false steps.

To that understanding, the swing classification engine 220 may be configured to estimate the first variation in the arm swing angle and the second variation in the leg swing angle. The first variation and the second variation may be due to one or more of the gait abnormality, a surface on which the walking activity is being performed, instability, fatigue, different left/right strides while performing the walking activity. In one embodiment, the swing classification engine 220 may further be configured to determine a time taken between a swing of an arm and a time taken between a swing of a leg and a distance travelled by the user during each swing.

In one embodiment, the compensation value calculation engine 222 may be configured to calculate a compensation value associated with the one or more false steps. The compensation value calculation engine 222 may be configured to use the ML model from a number of models for calculating the compensation value. Furthermore, the compensation value may be calculated by the ML model based on a combination of the first variation in the arm swing angle, the second variation in the leg swing angle, a height of the user, the time taken between the swing of the arm and the time taken between the swing of the leg, and a distance travelled by the user during each swing. The ML model may be selected based on the detection of the gait abnormality and the physical aid used by the user. The height of the user may be received as input from the user.

Continuing with the above embodiment, the step detection engine 224 may be configured to determine the step count of the user during the walking activity. The step count may be determined by applying the calculated compensation value to an initial step count of the user. The compensated value may be applied based on adjusting a maximum value and a minimum value for the threshold crossing value based on the compensation value.

For determining the initial step count, the determination engine 214 may be configured to determine a walking pattern associated with the user. The walking pattern may be determined based on the gait abnormality, the arm swing angle, and the leg swing angle. In one embodiment, the compensation value calculation engine 222 may be configured to estimate a walking cycle of the user based on the determined walking pattern. The walking cycle may be estimated by determining a threshold crossing value associated with the arm swing angle and the leg swing angle to determine each step included in the initial step count. Furthermore, the step detection engine 224 may be configured to determine the initial step count based on the estimated walking cycle.

In an embodiment, at least one of the determination engine 214, the abnormality detection engine 216, the aid detection engine 218, the swing classification engine 220, the compensation value calculation engine 222, or the step detection engine 224 may be included in the processor 204 as one or more program.

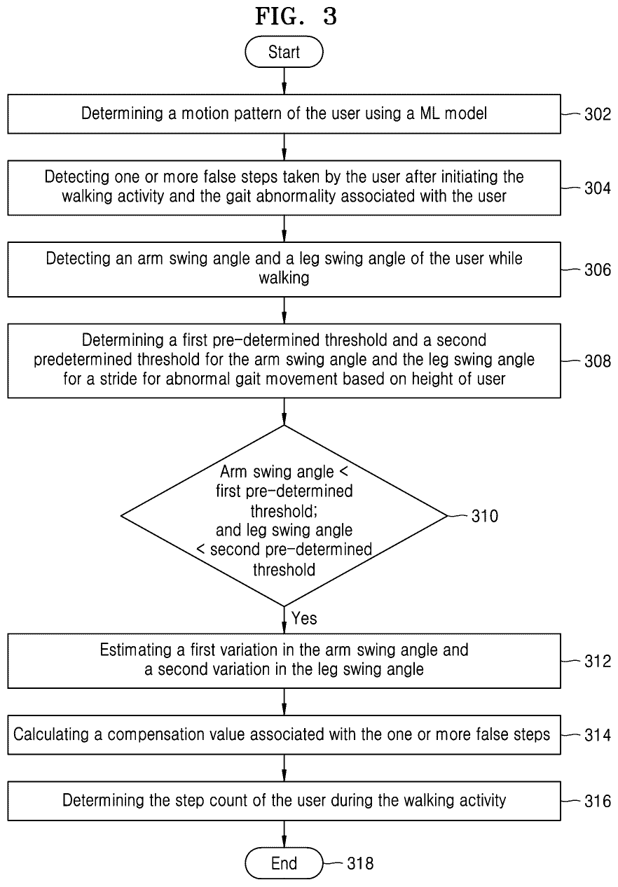

FIG. 3 illustrates a process for determining a step count of a user, in accordance with an embodiment of the disclosure. The process for determining the step count may be performed by the system 202 as referred in FIG. 2. The user may be performing a walking activity such as walking, pacing or the like. The user may be suffering from a physical disability or a disorder. Further, the user may be assisted with a physical aid while performing the walking activity. Examples of the physical aid may include, but are not limited to, a crutch, a leg brace, a walker, or canes.

At operation 302, the process may include determining a motion pattern of the user while performing the walking activity. The motion pattern may be determined by the determination engine 214 as referred in FIG. 2 by using a ML model. The motion of the user may indicate a posture of the user while walking, hand moments, feet moments, and a size for each step taken by the user after initiating the walking activity. The motion pattern of the user may be determined after the initiation of the walking activity by the user. Prior to determining the motion pattern, the process may include receiving by the determination engine 214 a notification indicating that the user is performing the walking activity. The notification may be transmitted by one or more sensors to the determination engine 214 in response to detecting that the user is in motion and performing the walking activity. Further, the one or more sensors may be one of an accelerometer, and a gyroscope. The one or more sensors may be incorporated within the electronic device. In an embodiment, the one or more sensors may be wearable sensors and may be worn by the user. In one embodiment, the one or more sensors may be present in a vicinity of the user.

At operation 304, the process may include detecting one or more false steps taken by the user after initiating the walking activity and the gait abnormality associated with the user. The detection may be performed by the abnormality detection engine 216 as referred in FIG. 2. The one or more false steps and the gait abnormality may be detected based on the motion pattern of the user while performing the walking activity. Examples of the gait abnormality may include, but are not limited to, a spastic gait, a scissors gait, a steppage gait, a waddling gait, a propulsive gait, an abnormality due to an amputation, and a biological abnormality.

Based on detection of the gait abnormality, the process may include detecting the physical aid used by the user during the walking activity based on the detection of the gait abnormality. The physical aid used by the user may be detected by the aid detection engine 218 as referred in FIG. 2.

At operation 306, the process may include detecting an arm swing angle of the user and a leg swing angle of the user while performing the walking activity. The arm swing angle and the leg swing angle may be dependent on a physical ability of the user. The arm swing angle of the user may be detected for one or more arms of the user and the legs swing angle may be detected for one or more legs of the user. The arm swing angle and the leg swing angle may be detected based on the motion pattern of the user, the gait abnormality associated with the user, the walking aid used by the user, and sensor data received from the one or more sensors.

To that understanding, the sensor data may be utilized to identify an intensity of the walking activity and a speed with which the walking activity is being performed. An increase in the intensity of the walking activity may increase the arm swing angle and the leg swing angle. The arm swing angle and the leg swing angle may be detected by the swing classification engine 220 as referred in FIG. 2. In one embodiment, the process may include utilizing a Multiple Linear Regression (MLR) technique to determine the arm swing angle and the leg swing angle.

At operation 308, the process may include determining a first pre-determined threshold value associated with the arm swing angle and a second pre-determined threshold value associated with the leg swing angle. The first pre-determined threshold value and the second pre-determined threshold value may be determined by the swing classification engine 220 as referred in FIG. 2. The first pre-determined threshold value and the second pre-determined threshold value may be determined based on a height of the user and the physical aid assisting the user while performing the walking activity.

At operation 310, the process may include determining whether the arm swing angle is less than the first pre-determined threshold value and the leg swing angle is less than the second pre-determined threshold value. The determining may be performed by the swing classification engine 220 as referred in FIG. 2. In an embodiment, where it is determined that the arm swing angle is less than the first pre-determined threshold value and the leg swing angle is less than the second pre-determined threshold value, the process may proceed towards operation 312. In an embodiment, where it is determined that the arm swing angle is not less than the first pre-determined threshold value and/or the leg swing angle is not less than the second pre-determined threshold value, the arm swing angle and the leg swing angle may be considered as a false entry.

At operation 312, the process may include estimating a first variation in the arm swing angle and a second variation in the leg swing angle. The estimation may be performed by the swing classification engine 220. The first variation and the second variation may be due to one or more of the gait abnormality, a surface on which the walking activity is being performed, instability, fatigue, different left/right strides while performing the walking activity. Further, the process may include determining a time taken between a swing of an arm and a swing of a leg, and a distance travelled by the user during each swing. The determining may be performed by the swing classification engine 220.

At operation 314, the process may include calculating a compensation value associated with the one or more false steps. The calculation may be performed by the compensation value calculation engine 222 as referred in FIG. 2. The compensation value may be calculated by using a ML model from a number of models for calculating the compensation value based on a combination of the first variation in the arm swing angle, the second variation in the leg swing angle, the height of the user, the time taken between the swing of the arm and the swing of the leg, and a distance travelled by the user during each swing. The ML model may be selected based on the detection of the gait abnormality and the physical aid used by the user. The compensation value may be calculated for intermediate swing positions of an arm and a leg of the user while the user is performing the walking activity. The compensation value may correspond to a compensation in an arm length, the arm swing angle, the leg swing angle, and the height of the user using the physical aid for performing the walking activity.

At operation 316, the process may include determining the step count of the user during the walking activity. The determination may be performed by the step detection engine 224. The step count may be determined by applying the calculated compensation value to an initial step count of the user. The compensated value may be applied based on adjusting a maximum value and a minimum value for the threshold crossing value.

For determining the initial step count, the process may include determining a walking pattern associated with the user. The determining of the walking pattern may be performed by the determination engine 214. The walking pattern may be determined based on the gait abnormality, the arm swing angle, and the leg swing angle. In one embodiment, the process may include estimating (e.g., by the compensation value calculation engine 222) a walking cycle of the user based on the determined walking pattern. The walking cycle may be estimated by determining a threshold crossing value associated with the arm swing angle and the leg swing angle to determine each step included in the initial step count. Furthermore, the process may include determining (e.g., by the step detection engine 224) the initial step count based on the estimated walking cycle.

At operation 318, the process may terminate.

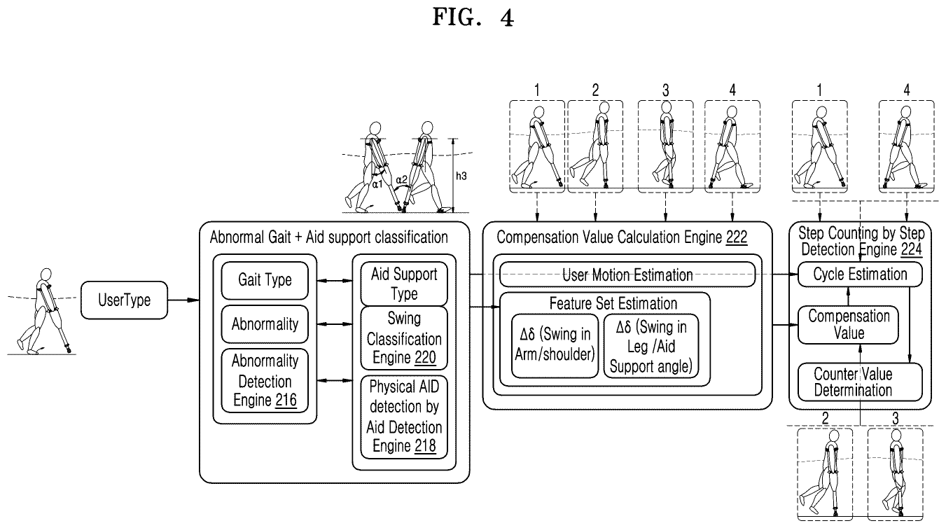

FIG. 4 illustrates an architectural diagram depicting a method for determining a step count of a user, in accordance with an embodiment of the disclosure. The method may be performed by the system 202 as referred in FIG. 2. The architecture may include the abnormality detection engine 216, the swing classification engine 220, the aid detection engine 218, the compensation value calculation engine 222, and the step detection engine 224 as referred in FIG. 2. The user may be performing a walking activity with a support of a physical aid. Examples of the physical aid may include, but are not limited to, a crutch, a leg brace, a walker, and canes.

Continuing with the above embodiment, the abnormality detection engine 216 may be configured to identify one or more false steps, and a gait abnormality associated with the user while the user performing the walking activity. The gait abnormality may be identified based on a motion pattern of a user. The motion of the user may indicate a posture of the user while walking, hand moments, feet moments, and a size for each step taken by the user after initiating the walking activity.

In one embodiment, the aid detection engine 218 may be configured to detect the physical aid used by the user during the walking activity based on the detection of the gait abnormality. Subsequently, the swing classification engine 220 may be configured to detect an arm swing angle of the user and a leg swing angle of the user while performing the walking activity. The arm swing angle and the leg swing angle may be detected based on the motion pattern of the user, the gait abnormality associated with the user, the walking aid used by the user, and sensor data received from the one or more sensors.

Continuing with the above embodiment, the swing classification engine 220 may further be configured to determine a first pre-determined threshold value associated with the arm swing angle and a second pre-determined threshold value associated with the leg swing angle. Further, if it is determined that the arm swing angle is less than the first pre-determined threshold value and the leg swing angle is less than the second pre-determined threshold value, the swing classification engine 220 may be configured to estimate a first variation in the arm swing angle and a second variation in the leg swing angle and a time taken between a swing of an arm and a swing of a leg and a distance travelled by the user during each swing. The first variation and the second variation may be due to one or more of the gait abnormality, a surface on which the walking activity is being performed, instability, fatigue, different left/right strides while performing the walking activity.

To that understanding, the compensation value calculation engine 222 may be configured to calculate a compensation value associated with the one or more false steps based on a ML model. The ML model may be selected based on the detection of the gait abnormality and the physical aid used by the user. The ML model may be calculated based on a combination of the first variation in the arm swing angle, the second variation in the leg swing angle, a height of the user, the time taken between the swing of the arm and the swing of the leg, and a distance travelled by the user during each swing.

Continuing with the above embodiment, the step detection engine 224 may be configured to determine the step count of the user during the walking activity by applying the calculated compensation value to an initial step count of the user. The compensated value may be applied by adjusting a maximum value and a minimum value for the threshold crossing value based on the compensation value.

For determining the initial step count, the determination engine 214 may be configured to determine a walking pattern associated with the user. In one embodiment, the compensation value calculation engine 222 may be configured to estimate a walking cycle of the user based on the determined walking pattern by determining a threshold crossing value associated with the arm swing angle and the leg swing angle to determine each step included in the initial step count. Furthermore, the step detection engine 224 may be configured to determine the initial step count based on the estimated walking cycle. Based on the initial step count, the step count of the user may be determined.

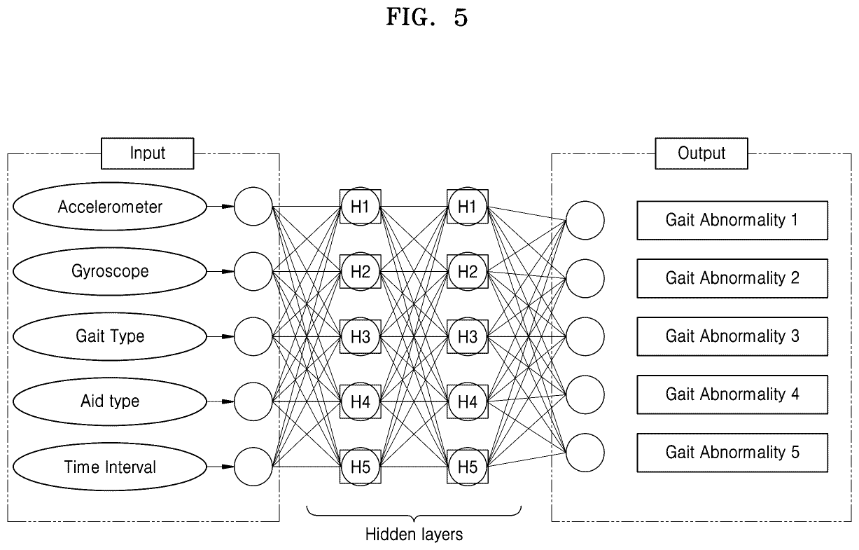

FIG. 5 illustrates a process for determining a gait abnormality associated with the user, in accordance with an embodiment of the disclosure. The gait abnormality may be determined when the user is performing a walking activity. The gait abnormality may be determined by the abnormality detection engine 216 as referred in FIG. 2 by utilizing an Artificial Neural Network (ANN). The gait abnormality may be detected based on a motion pattern of the user while performing the walking activity. Examples of the gait abnormality may include, but are not limited to, a spastic gait, a scissors gait, a steppage gait, a waddling gait, a propulsive gait, an abnormality due to an amputation, and a biological abnormality.

The spastic gait may occur when the user drags feet while performing the walking activity, making the user appear to be stiff when performing the walking activity. The scissors gait is associated with the bending of legs inward such that legs of the user with the scissors gait may cross and may hit one another while performing the walking activity. The steppage gait may occur toes of the user point towards ground the user is walking. The toes may scrape against the ground as the user steps forward. Furthermore, the waddling gait may cause the user to move from side to side while walking. Waddling includes taking short steps as well as swinging the body. Also, the propulsive gait may cause the user to walk with head and neck pushing forward. The user with the propulsive gait may appear as though rigidly holding a slouched position.

The process may include using the ANN trained with backpropagation to identify a walk pattern of the user. Further, the process may include providing the ANN a number of inputs such as sensor data related to one or more sensors, gait abnormality associated with the user, a physical aid assisting the user, a time interval for regular seen pattern. The sensor data may include accelerometer readings, and/or gyroscope readings.

Further, the ANN may be trained using data collected from wide a range of users suffering through one of the gait abnormalities using the physical aid for which abnormality type is known. The identified walk pattern may be later used to detect steps by applying related threshold on the sensor data to detect steps taken by the user.

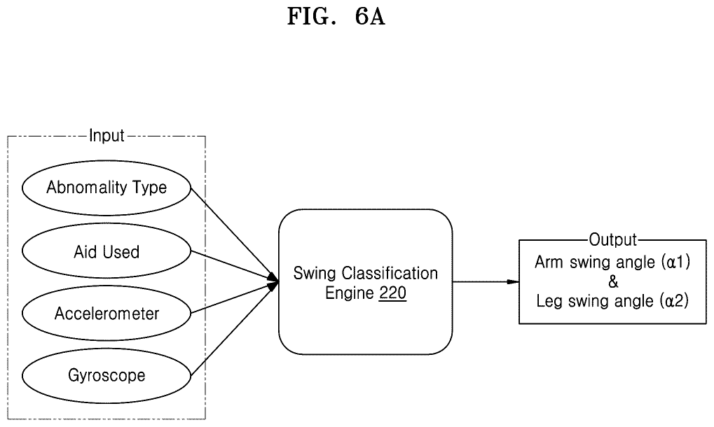

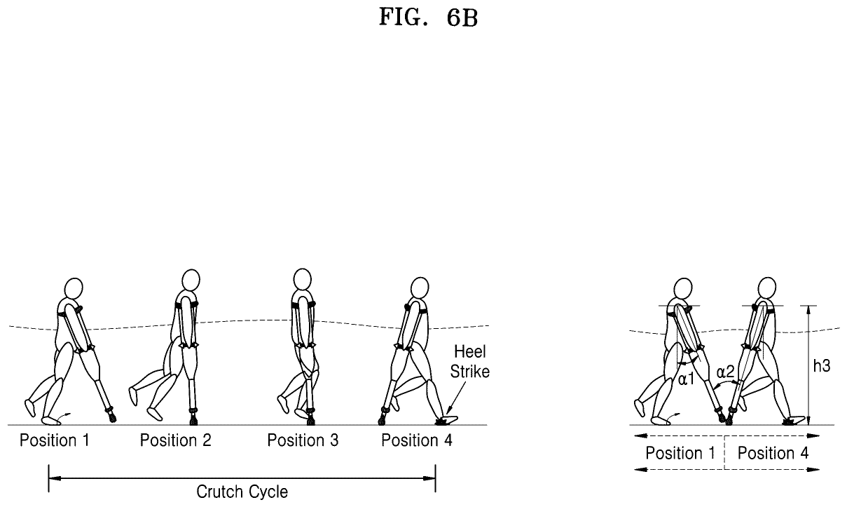

FIG. 6A illustrates a process for determining an arm swing angle of a user and a leg swing angle of a user, in accordance with an embodiment of the disclosure. The determination may be performed by the swing classification engine 220 as referred in FIG. 2. The swing classification engine 220 may be a supervised trained engine and may be configured to detect the arm swing angle and the leg swing angle for each step taken by the user. The arm swing angle and the leg swing angle may be determined while the user is performing a walking activity.

The arm swing angle and the leg swing angle may be dependent on a physical ability of the user. The user with a gait abnormality may demonstrate different physical capabilities and the different physical capabilities may impact the arm swing angle and the leg swing angle. The arm swing angle and the leg swing angle may be detected based on a motion pattern of the user, a gait abnormality associated with the user, a physical aid used by the user as each physical aid may require a unique usage style that may affect the arm swing angle and the leg swing angle, and sensor data associated with the walking activity received from one or more sensors.

To that understanding, the sensor data may be utilized to identify an intensity of the walking activity and a speed with which the walking activity is being performed. An increase in the intensity of the walking activity may increase the arm swing angle and the leg swing angle. In one embodiment, the process may include determining the arm swing angle and the leg swing angle by using a MLR technique. The arm swing angle and the leg swing angle may be determined based on equation 1 below:

$$Y = {β}_{0} + {β}_{1} {x}_{1} + {β}_{2} {x}_{2} + … + {β}_{p} {x}_{p} + ε \qquad [ Equation 1 ]$$

Wherein the ‘y-variable’ Y is a response, a dependent variable, and an observation. The ‘x-variable’ x is a predictor, an independent variable, and an explanatory variable. The β is a coefficient. The term β₀+B₁x₁+B₂x₂+ . . . +βₚxₚ is a linear predictor, and the term ε is a noise or a random error.

The swing classification engine 220 may be configured to utilize the MLR technique and find a relationship between at least two independent variables (inputs) and corresponding dependent variable may be estimated as an output as depicted in table 1. The corresponding dependent variable may be the arm swing angle or the leg swing angle. For determining the arm swing angle and the leg swing angle, the swing classification engine 220 may be trained with a large range of data input sets such that a correct output is predicted with least error. Further, the MLR technique may be used to train the swing classification engine 220 based on the gait abnormality, the physical aid used, and the sensor data associated with the one or more sensors.

TABLE 1

| | | | | | Output | Output |
|---|---|---|---|---|---|---|
| | | | | | Estimated | Estimated |
| | Abnormality | Walking Aid | Accelerometer | Gyroscope | Arm Swing | Leg Swing |
| Input | Type X1 | Type X2 | X3 | X4 | Angle Y1 | Angle Y2 |
| #1 | 3 | 2 | 50 | 25 | 31 | 56 |
| #2 | 1 | 2 | 60 | 10 | 27 | 41 |
| #3 | 5 | 1 | 53 | 13 | 39 | 61 |
| #4 | | | | | | |

Table 1 depicts input utilized to determine the arm swing angle or leg swing angle.

FIG. 6B illustrates a diagram depicting a crutch cycle of a user while performing the walking activity, in accordance with an embodiment of the disclosure. The crutch cycle may include a number of positions attained by the user while performing the walking activity. The number of positions may be repeated by the user for walking each step. The number of positions may include a movement of arms and legs of the user. The arms and legs of the user may move at a certain angle also referred as the arm swing angle for the arms and the leg swing angle for the legs while moving forward with the physical aid. In an example embodiment, the arm swing angle may be made by arms from a vertical position while performing the walking activity and the leg swing angle may be covered during a leg swing motion in a complete crutch cycle. In FIG. 6B, the arm swing angle may be depicted as α1 and the leg swing angle may be depicted as α2. The arm swing angle of the user may be less than a first pre-determined threshold and the leg swing angle of the user may be less than a second pre-determined threshold for a stride associated with one or more false steps due to the gait abnormality of the user that are not within an average dynamic threshold (maximum and minimum) of a pedometer within a time frame.

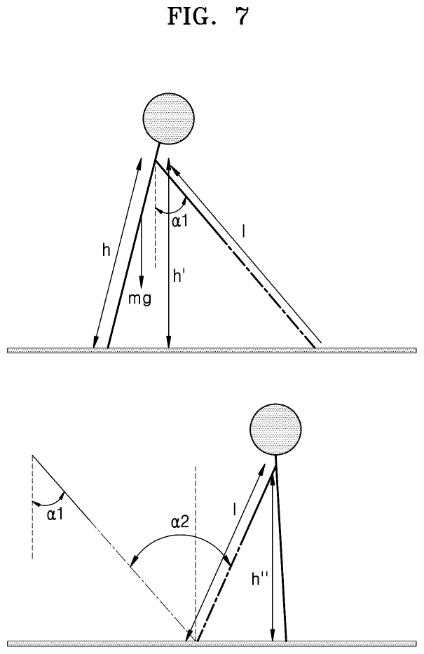

FIG. 7 illustrates a process for determining a first pre-determined threshold value associated with an arm swing angle and a second pre-determined threshold value associated with a leg swing angle of a user, in accordance with an embodiment of the disclosure. The arm swing angle and the leg swing angle may be determined when the user is performing a walking activity. The first pre-determined threshold value and the second pre-determined threshold value may be determined by the swing classification engine 220 as referred in FIG. 2. The first pre-determined threshold value and the second pre-determined threshold value may be applied on the arm swing angle and the leg swing angle, respectively, to eliminate unwanted wrong detections. The first pre-determined threshold value and the second pre-determined threshold value may be determined based on a height of the user and the physical aid assisting the user while performing the walking activity.

The user walking with the physical aid may extend an arm and lean forward to a certain limit without slipping or falling due to gravitational force, height, and a fixed length of the physical aid. The fixed length of the physical aid may depend on the physical aid being used by the user and may vary for each physical aid. In an example embodiment, where a shoulder height of the user is “h” and the fixed length of the physical aid including arms is “l”, the user may only lean forward about 20 degrees due to torque on body weight of the user. Hence, the shoulder height after leaning, h′ may be “h sin 20°”. Therefore, if a swing of the arm is denoted as α1, then cos α1=h′/l. Further, a user may lean backward only up to 10 degrees, therefore, the shoulder height leaning backward, “h″=h sin 10°”. Hence if a swing of the leg is denoted by α2, then cos(90−α2+α1)=h″/l (i.e., sin(α2−α1)=h″/l).

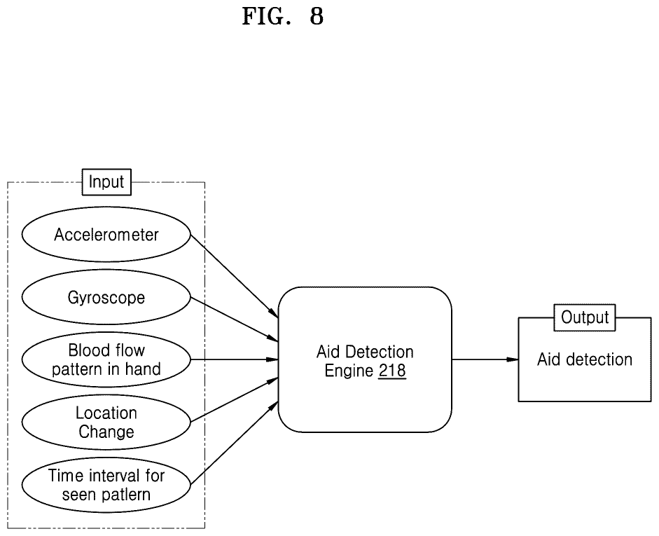

FIG. 8 illustrates a process for detecting a physical aid used by a user while performing a walking activity, in accordance with an embodiment of the disclosure. The physical aid used by the user may be detected by the aid detection engine 218 as referred in FIG. 2. The aid detection engine 218 may be trained to classify events to determine an instant to detect the physical aid used by the user using a classification technique based predictive modelling. The classification technique may be a Naive Bayes classification technique as depicted in equation 2 and equation 3 mentioned below:

$$P ( Found \vert X 1 , X 2 , X 3 , X 4 , X 5 ) = \frac{\begin{matrix}P ( X 1 \vert Found ) * P ( X 2 \vert Found ) * P ( X 3 \vert Found ) * \\ P ( X 4 \vert Found ) * P ( X 5 \vert Found )\end{matrix}}{P ( X 1 , X 2 , X 3 , X 4 , X 5 )} \qquad [ Equation 2 ]$$

$$P ( NotF ound \vert X 1 , X 2 , X 3 , X 4 , X 5 ) = \frac{\begin{matrix}P ( X 1 \vert NotFound ) * P ( X 2 \vert NotFound ) * \\ P ( X 3 \vert NotFound ) * P ( X 4 \vert NotFound ) * \\ P ( X 5 \vert NotFound )\end{matrix}}{P ( X 1 , X 2 , X 3 , X 4 , X 5 )} \qquad [ Equation 3 ]$$

The aid detection engine 218 may be trained using supervised learning with a number of parameters as a feature matrix and an expected outcome of the feature matrix as a response vector as depicted in table 2. The response vector may indicate whether the physical aid is used. The number of parameters may include accelerometer data, gyroscope data, a blood flow pattern, a location, and a time interval data associated with the user.

TABLE 2

| | | | | | Aid |
|---|---|---|---|---|---|
| | | Blood | Location | | Detection |
| Accelerometer | Gyroscope | Flow | Change | Time | State |
| Pattern | Pattern | Pattern | Pattern | Interval | (Found/Not |
| (X1) | (X2) | (X3) | (X4) | (X5) | Found) |
| 0 | 1 | 0 | 0 | 1 | Found |
| 1 | 0 | 1 | 0 | 0 | Not Found |
| 0 | 0 | 1 | 1 | 0 | Found |
| 0 | 1 | 0 | 1 | 1 | Found |
| 0 | 0 | 0 | 1 | 0 | Not Found |

Table 2 depicts a number of parameters used to determine a usage of the physical aid by the user.

In an example embodiment, when the user is using crutches as the physical aid to walk, the user may keep a smartphone of the user in a pocket due to lack of holding space for the smartphone in hand of the user. Further, whenever the user takes a swing on the crutches, a higher spike on accelerometer may be detected as opposed to when a user is waling without the physical aid during a normal walk. In one example embodiment, when pressure is applied on the hand or a weight is being carried via the hand, a change in blood flow towards wrist and palm may occur. By using a light sensor, a difference may be measured as a red light of the light sensor penetrates through skin of the user and goes in blood particles depending upon a blood flow and a density of the blood. Some part of the red light may be absorbed, and a remaining part may be reflected back to a receiver in the light sensor. Further, the difference in concentration or flow of blood into the wrist may be measured.

In yet one embodiment, where the user put a weight of the user on crutches, a change in blood flow towards wrist may occur. If a user movement is observed, it may be ascertained that there is always a certain time interval after which the user applies a pressure on the crutches. The time interval may be used as one of the number of parameters. Further, a change in location data may occur while the user is using the crutches while walking, the location data may be used as an indicator.

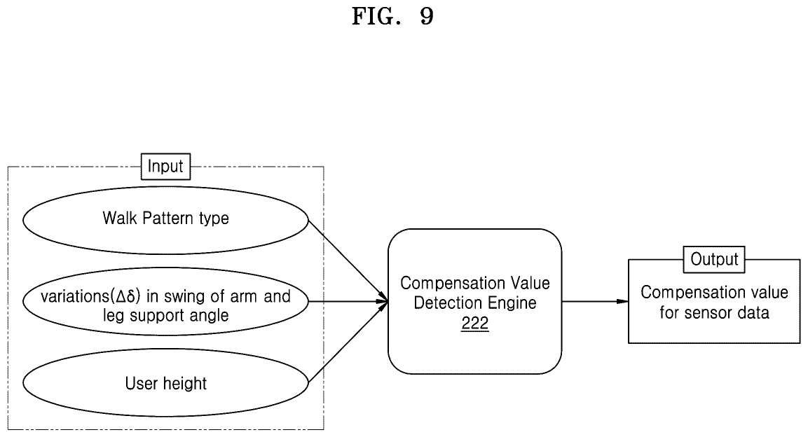

FIG. 9 illustrates a process for calculating a compensation value associated with one or more false steps of a user, in accordance with an embodiment of the disclosure. The compensation value may be calculated by the compensation value calculation engine 222 as referred in FIG. 2. The compensation engine may utilize supervised machine learning to determine the compensation value to be applied to detect correct steps from sensor data associated with the user.

Furthermore, while walking with a physical aid, the user may experience a variation in a step length due to surface ahead, instability, fatigue, different left/right strides. The variation may cause a change (Δ∂) in an arm swing angle and a leg swing angle of the user. The change may be utilized for correctly determining a step count of the user and a distance covered by the user. The compensation value calculation engine 222 may utilize a change in angles of the physical aid such as a crutch to determine the compensation value on a maximum/minimum for a step detection to determine the step count from the sensor data. The step count may be corrected for variable step sizes by applying the compensation value.

The compensation value calculation engine 222 may utilize a MLR technique to find a relationship between at least two independent variables (inputs) and corresponding dependent variable may be estimated as an output and the output may be the compensation value as depicted in table 3. The arm swing angle and the leg swing angle may be determined based on the equation 1 mentioned above.

The compensation value calculation engine 222 may be trained with a large range of data input sets such that a correct output is predicted with least error. Further, the MLR technique may be used to train the compensation value calculation engine 222 based on features such as, a change in the arm swing angle, a change in the leg swing angle, a walk pattern type, and a height of the user.

TABLE 3

| | Change in | Change in | Walk | | Output |
|---|---|---|---|---|---|
| | arm swing | leg swing | Pattern | User | compensation |
| | angle | angle | Type | Height | value |
| Input | X1 | X2 | X3 | X4 | Y2 |
| #1 | 15 | 20 | 3 | 170 | 56 |
| #2 | 7 | 2 | 1 | 155 | 41 |
| #3 | −4 | 1 | 5 | 162 | 61 |
| #4 | | | | | |

Table 3 depicts input utilized to determine the compensation value.

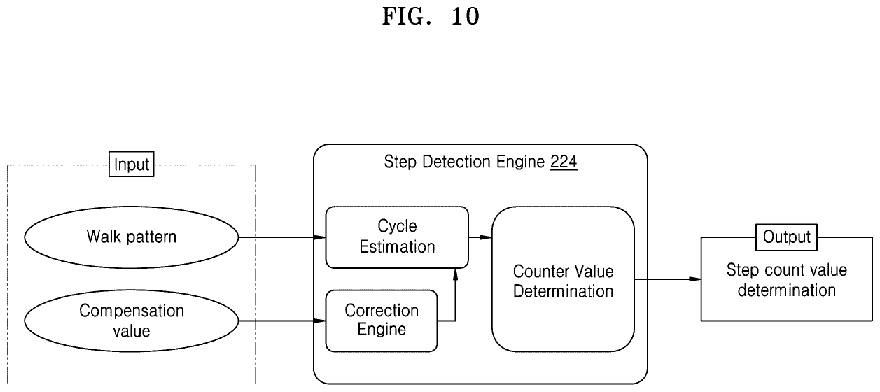

FIG. 10 illustrates a process for determining step counts of a user based on a compensation value, in accordance with an embodiment of the disclosure. The compensation value may be calculated by the compensation value calculation engine 222 as referred in FIG. 2. The process may be performed by the step detection engine 224 as referred in FIG. 2. The step count may be determined by applying the calculated compensation value to an initial step count of the user based on adjusting a maximum value and a minimum value for a threshold crossing value.

The initial step count may be determined based on a walking pattern associated with the user (e.g., by the determination engine 214 as referred in FIG. 2). The walking pattern may be determined based on the gait abnormality, the arm swing angle, and the leg swing angle. For determining the initial step count, the process may include estimating a walking cycle of the user (e.g., by the compensation value calculation engine 222) based on the walking pattern. Estimating the walking cycle may include determining the threshold crossing value associated with an arm swing angle and a leg swing angle of the user to determine each step included in the initial step count.

While specific language has been used to describe the disclosure, any limitations arising on account thereto, are not intended. As would be apparent to a person in the art, various working modifications may be made to the method in order to implement the inventive concepts as taught herein. The drawings and the foregoing description give examples of embodiments. Those skilled in the art will appreciate that the described elements may well be combined into a single functional element. Alternatively, certain elements may be split into multiple functional elements. Elements from one embodiment may be added to one embodiment. The disclosure may be otherwise variously embodied, and practiced within the scope of the following claims.

What is claimed is:

1. A method for determining a step count of a user, the method comprising:
determining a motion pattern of the user after an initiation of a walking activity by the user;
detecting one or more false steps and a gait abnormality associated with the user, based on the motion pattern;
detecting an arm swing angle of the user and a leg swing angle of the user, based on the motion pattern and the walking activity;
estimating a first variation in the arm swing angle and a second variation in the leg swing angle in the walking activity;
calculating, using a machine learning (ML) model of a plurality of ML models, a compensation value associated with the one or more false steps, based on a combination of the first variation and the second variation, and a height of the user; and
determining the step count of the user, based on the calculated compensation value and an initial step count of the user.

2. The method of claim 1, further comprising:
determining a time taken on a swing of an arm and a swing of a leg, and a distance travelled by the user during a swing; and
calculating the compensation value based on the determined time.

3. The method of claim 1, wherein the first variation and the second variation are determined based on at least one of the gait abnormality, a surface of the walking activity, instability, fatigue, one or more left strides of the user, and one or more right strides of the user.

4. The method of claim 1, wherein the calculating the compensation value comprises:
determining a first pre-determined threshold value associated with the arm swing angle and a second pre-determined threshold value associated with the leg swing angle;
determining whether the arm swing angle is less than the first pre-determined threshold value and the leg swing angle is less than the second pre-determined threshold value; and
calculating the compensation value based on a result of a determination that the arm swing angle is less than the first pre-determined threshold value and the leg swing angle is less than the second pre-determined threshold value.

5. The method of claim 4, further comprising detecting a physical aid used by the user during the walking activity, based on the gait abnormality,
wherein at least one of the first pre-determined threshold value and the second pre-determined threshold value is determined based on the height of the user and the physical aid used by the user during the walking activity.

6. The method of claim 1, further comprising:
determining a walking pattern associated with the user, based on the gait abnormality, the arm swing angle, and the leg swing angle;
estimating a walking cycle of the user, based on the determined walking pattern; and
determining the initial step count, based on the estimated walking cycle.

7. The method of claim 6, wherein the estimating the walking cycle of the user further comprises determining a threshold crossing value associated with the arm swing angle and the leg swing angle to determine at least one step in the initial step count.

8. The method of claim 7, further comprising adjusting a maximum value and a minimum value for the threshold crossing value, based on the compensation value.

9. The method of claim 1, wherein the gait abnormality comprises at least one of a spastic gait, a scissors gait, a steppage gait, a waddling gait, a propulsive gait, an abnormality due to an amputation, or a biological abnormality.

10. The method of claim 1, wherein the ML model is selected, based on the gait abnormality and a physical aid used by the user.

11. A system for determining a step count of a user, the system comprising:
a determination engine configured to determine a motion pattern of a user after initiation of a walking activity by the user;
an abnormality detection engine configured to detect one or more false steps and a gait abnormality associated with the user, based on the motion pattern;
a swing classification engine configured to:
detect an arm swing angle of the user and a leg swing angle of the user, based on the motion pattern in the walking activity; and
estimate a first variation in the arm swing angle and a second variation in the leg swing angle in the walking activity;
a compensation value calculation engine configured to calculate, using a Machine Learning (ML) model of a plurality of ML models, a compensation value associated with the one or more false steps, based on a combination of the first variation and the second variation, and a height of the user; and
a step detection engine configured to determine the step count of the user, based on the calculated compensation value and an initial step count of the user.

12. The system of claim 11, wherein the swing classification engine is further configured to determine a time taken on a swing of an arm and a swing of a leg, and a distance travelled by the user during a swing; and
wherein the compensation value calculation engine is further configured to calculate the compensation value, based on the determined time.

13. The system of claim 11, wherein the calculating the compensation value comprises:
determining a first pre-determined threshold value associated with the arm swing angle and a second pre-determined threshold value associated with the leg swing angle;
determining whether the arm swing angle is less than the first pre-determined threshold value and the leg swing angle is less than the second pre-determined threshold value; and
calculating the compensation value based on a result of the determination that the arm swing angle is less than the first pre-determined threshold value and the leg swing angle is less than the second pre-determined threshold value.

14. The system of claim 13, further comprising an aid detection engine configured to detect a physical aid used by the user during the walking activity, based on the gait abnormality,
wherein at least one of the first pre-determined threshold value and the second pre-determined threshold value is determined based on the height of the user and the physical aid used by the user during the walking activity.

15. The system of claim 11, wherein the determination engine is further configured to determine a walking pattern associated with the user based on the gait abnormality, the arm swing angle, and the leg swing angle;
wherein the compensation value calculation engine is further configured to estimate a walking cycle of the user based on the determined walking pattern; and
wherein the step detection engine is further configured to determine the initial step count based on the estimated walking cycle.
